(19) United States
(12) Patent Application Publication
(10) Pub. No.: US 2005/0123438 A1
(43) Pub. Date: Jun. 9, 2005

(54) Method of preparing material

(76) Inventor: Bruno Aubert, Connaux (FR)

Correspondence Address: FOLEY AND LARDNER;SUITE 500, 3000 K STREET NW, WASHINGTON, DC, 20007, US

(21) Appl. No.: 10/505,222

(22) PCT Filed: Feb. 13, 2003
(86) PCT No.: PCT/EP03/50016

(30) Foreign Application Priority Data
Feb. 20, 2002 (EP) 02100153.2

(51) Int. Cl. A61L 2/02; B29C 43/02

(52) U.S. Cl. 422/28; 264/320; 264/322; 422/26; 422/33

(57) ABSTRACT

The invention relates to a method of preparing material to be further subjected to a heat and/or compaction treatment. The method comprises the step of—introducing the material in a plastic recipient (20) having an inner layer and an outer layer. The inner layer has a melting point lower than the temperature reached during the heat treatment, whereas the outer layer has a melting point higher than the temperature reached during the heat treatment.—closing the plastic recipient in such a way that the inner layer is not appearing at the outer surface of the closed recipient. The invention further relates to a method of disinfecting or sterilizing material.

### FIELD OF THE INVENTION

The invention relates to a method of preparing material to be further subjected to a heat and/or compaction treatment. The invention further relates to a method of disinfecting or sterilising material.

### BACKGROUND OF THE INVENTION

Before waste, such as medical waste, is further treated, for example disinfected or sterilised, generally it is collected in a bag or box, as for example a polyethylene bag.

Once these bags are filled they can be transported to an installation for further treatment such as an autoclave, an incinerator or a disinfecting device.

Medical waste has a very low density and thus a high volume. Because of this high volume of the bags the transportation of the waste to an installation for further treatment is expensive. Reduction of the volume of the waste is thus desired. However, the reduction of the volume of the medical waste is rather difficult since the medical waste comprises a lot of elastic material such as cotton, gloves, . . . Often after compaction, the waste recovers the original volume just like a spring.

### SUMMARY OF THE INVENTION

It is an object of the present invention to provide a method of preparing a material to be further subjected to a heat and/or compaction treatment.

It is another object of the invention to provide a method of preparing material to be further treated in a safe and efficient way.

According to a first aspect of the present invention, a method of preparing material to be subjected to a heat treatment and possibly also to a compaction treatment is provided.

The method comprises the steps of
introducing the material in a plastic recipient having an inner layer and an outer layer. The inner layer has a melting point lower than the temperature reached during the heat treatment, whereas the outer layer has a melting point higher than the temperature reached during the heat treatment.
closing the plastic recipient in such a way that the inner layer is not appearing at the outer surface of the closed recipient.

The plastic recipient may for example comprise a plastic box or bag. Either the inner layer as the outer layer comprise a plastic material.

For disinfecting applications, the melting point of the inner layer will be lower than the disinfecting temperature which is preferably between 121° C. and 150° C.

Preferably, the melting point of the inner layer is lower than 100° C., for example between 80° C. and 100° C.

In principle any material that has a melting point lower than 100° C. can be used as inner layer.

The material of the inner layer is for example selected from the group consisting of polyethylene, polycarbonate (PC), polyphenyleneoxide or modified polyphenyleneoxide (PPO), polymethylmethacrylate (PMMA) and polyoxymethylene copolymer.

In a preferred embodiment, the inner layer comprises polyethylene such as high or low density polyethylene.

Since the inner layer has a melting point which is lower than the temperature reached during the heat treatment, it is clear that the inner layer will melt during the heat treatment.

This melted layer will stick to the material collected inside the plastic recipient and will ensure a good adhesion with the material.

If the plastic recipient with the collected material is compacted, either before, during or after the heat treatment, the adhesion of the inner layer with the material collected in the plastic recipient will ensure that the material will keep its compacted form after the compaction step.

The outer layer has a melting point which is higher than the melting point of the inner layer.

If the material is subjected to a heat treatment, the outer layer has a melting point which is higher than the temperature reached during the heat treatment.

For disinfecting applications, the melting point of the outer layer will be higher than the disinfecting temperature (121° C. to 150° C.). Preferably, the melting point of the outer layer is higher than 160° C., for example between 160° C. and 200° C.

In principle any material that has a melting point higher than the disinfecting temperature can be used as outer layer.

The material of the outer layer is for example selected from the group consisting of polyamide (PA), polyethyleneterephtalate (PET), silicon, polyvinylidene fluoride (PVDF), polytetrafluoroethylene (PTFE) for example known as teflon®, polyaramide, polyimide (PI) for example known as Kapton® or Upilex®, polyetherimide (PI), fluorinated ethylene propylene copolymer (FEP), ethylene tetrafluoroethylene copolymer (ETFE), hexafluoropropylenevinylidenefluoride copolymer (FKM) and tetrafluoroethylene-perfluoro(alkoxy vinyl ether) copolymer.

In a preferred embodiment, the outer layer comprises a polyamide such as nylon 6, nylon 6.6 or nylon 6.10.

Since the melting point of the outer layer is higher than the temperature reached during the heat treatment, the outer layer will not melt during this heat treatment. This is important since adhesion of the outer layer with the device in which it is introduced has to be avoided.

After all, it is the purpose that the treated (compacted and disinfected) material can easily be removed from the device in which the heat treatment is performed.

To avoid any sticking of the plastic recipient to the interior of the device in which the heat treatment is performed, it is important that the plastic recipient is closed in such a way that the inner layer is not appearing at the outer surface of the closed recipient.

The plastic recipients can be closed in numerous ways. Plastic bags for example can be closed by folding the bag in such a way that the inner layer is not appearing at the outer surface of the plastic bag once closed. Another common way of closure is by folding a flap of one wall of a plastic bag over the opening of the plastic bag and by securing this flap to the other wall of the bag for example by glueing.

Plastic boxes can be closed by means of a lid.

It can be preferred that a lid of a plastic box is provided with an air tightness tap or valve.

Preferably, the multi-layered plastic recipient, having an inner and an outer layer, has a sufficient high strength so that tears of the plastic recipient can be avoided even in case sharp objects, such as for example needles or scalpels are collected in the plastic recipient.

It can be preferred that the material such as the medical waste is first collected in a conventional bag before being introduced in the special multi-layered recipient.

The thickness of the inner layer is preferably between 50 and 500 μm as for example 180 μm.

The thickness of the outer layer is preferably between 10 and 100 μm as for example 70 μm.

It is preferred that the thickness of the recipient is higher than the free space between different parts of the disinfecting device in which the recipient is introduced.

Possibly, the plastic recipient comprises one or more intermediate layer(s) between the inner layer and the outer layer.

The plastic recipients used in the method of the present invention may have any shape and any dimensions. Preferably, the shape of the plastic recipient, such as the plastic bag is adapted to the shape of the device in which it is introduced for heating and/or compacting.

The dimensions are preferably adapted to the dimensions of this device. In case the device has a cylindrical shape, the plastic bag has preferably a tubular shape that fits into this cylindrical shape although other shaped bags such as bags with a rectangular or square bottom surface or bags with other dimensions are not excluded.

It can be preferred that the plastic recipient is made opaque. This can for example be achieved by adding a metal oxide to one or more layers of the plastic recipient, for example to the inner layer.

In order to avoid any mistake between an infected and disinfected plastic recipient a thermal indicator such as a thermochromic additive can be added to one or more layers of the plastic recipient. Such a thermochromic additive permit to change the color of the recipient once the temperature treatment is performed successfully.

Furthermore, such a thermal indicator makes it clear for an operator that the disinfecting process is performed correctly.

Alternatively, a label comprising a thermal indicator such as a thermochromic additive can be adhered to a plastic recipient.

In order to facilitate the compaction of the plastic recipient it can be preferred that the plastic bag is provided with a valve.

The method according to the present invention is suitable to prepare all kind of material to be further treated in a process involving heat and/or compaction. The method is in particular suitable to prepare medical waste for further treatment such as a disinfecting or sterilising treatment. As medical waste all waste generated in physicians' offices or hospitals, such as cotton, gauze pads, gloves, blood soaked linen, blood tubes, needles, syringes, scalpel blades, blood vials, culture dishes, . . . can be considered.

According to a second aspect of the present invention a method of disinfecting or sterilising material which is prepared according to the above described method is provided.

The method comprises the steps of:
introducing the closed plastic recipient, holding the material to be disinfected in a disinfecting device;
subjecting the closed plastic recipient to a heat treatment and possibly also to a compaction treatment.

During the heat treatment, the reached temperature is preferably higher than the melting temperature of the inner layer.

The material to be disinfected is preferably heated under a water vapour and/or radical pressure. The water vapour an/or radical pressure is preferably higher than 1 bar, for example 2 bar or 3.4 bar.

The radicals are preferably hydroxyl (OH) radicals. OH radicals are known as very reactive free radicals and as strong oxidants which may kill diverse microorganisms and degrade diverse volatile organic compounds.

Possibly, the radicals comprise also CO radicals.

The water vapour pressure and/or radical pressure is obtained during the heating by evaporating the water and the radicals absorbed and/or formed in a natural way at the surface of the material to be disinfected.

In a preferred method the water vapour and/or radical pressure is obtained by eliminating, before the heating, substantially all the air and the free space in and around the material to be disinfected in order to allow the water and the radicals absorbed and/or formed in a natural way at the surface of the material to be sufficiently numerous to obtain a saturated vapour pressure during the heating of the material.

It is known that all materials in atmospheric conditions absorb a small quantity of water on their surface and that this water presents itself partly under a radical form due to the interaction with the material. In order to guarantee that the water and the radicals, such as OH, are present under the form of vapour, it is preferred that the air and free space in and around the material to be disinfected or sterilised is substantially eliminated.

With the substantial elimination of the air and free space in and around the material is meant that the volume of air and free space in and around the material to be disinfected is less than 20% and preferably less than 10% of the total volume of the material. More preferably, the volume of air and free space in and around the material is less than 5%, for example less than 2% of the total volume of the material. By the substantial elimination of the air and free space in and around the material to be disinfected, the water and the OH radical absorbed or naturally formed at the surface or in the porous structure of the material will, when they are completely or partly evaporated through the heating process at temperatures over 100° C., finally be highly concentrated in the very small remaining volume and the vapour or radicals and germs will very probably meet.

The action of such a radical-charged water vapour is extremely efficient to destroy germs.

A preferred method comprises:
introducing the closed plastic recipient, holding the material to be disinfected in a disinfecting device;
eliminating substantially all the air and free space that is present in and around said material to be disinfected;
establishing airtight conditions
creating a water vapour pressure and/or radical pressure by heating the receptacle and/or the material.

### BRIEF DESCRIPTION OF THE DRAWINGS

The invention will now be described into more detail with reference to the accompanying drawings wherein

FIGS. 1 and 2 show two different methods of closing a plastic bag according to the present invention;

FIG. 3 shows a plastic box according to the present invention.

### DESCRIPTION OF THE PREFERRED EMBODIMENTS OF THE INVENTION

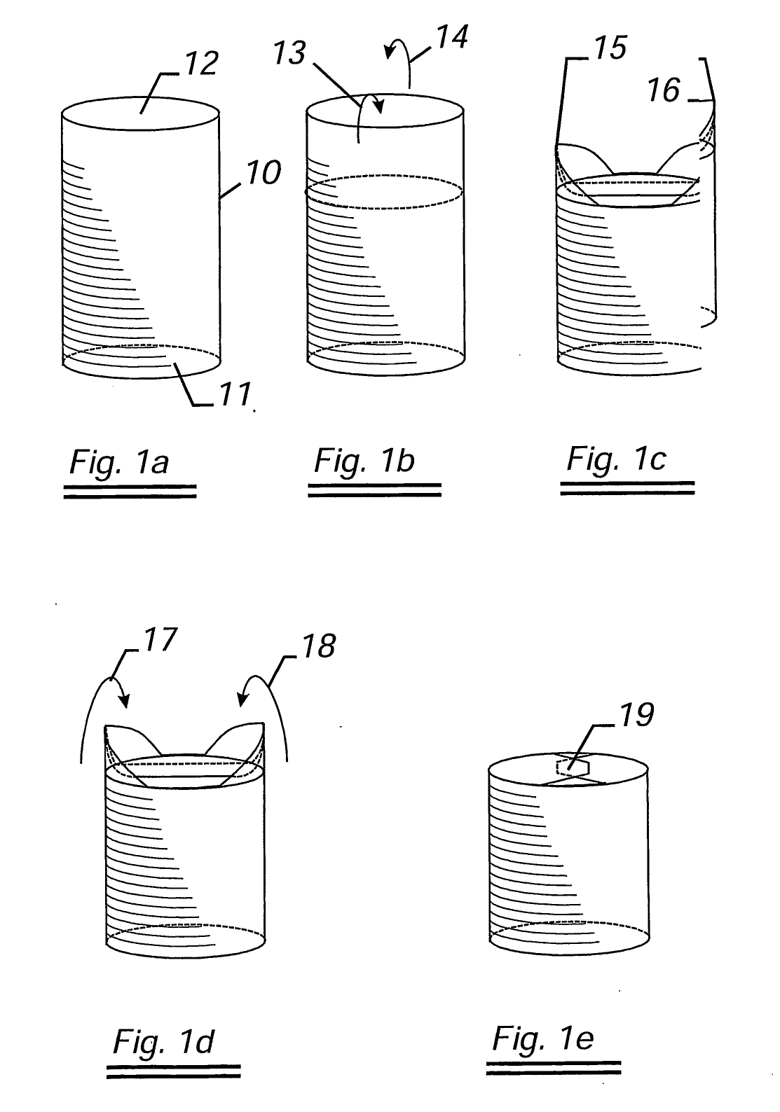

Referring to FIG. 1 a plastic bag 10 having a tubular shape is provided. The plastic bag 10 has a closed bottom end 11 and an open top end 12. The plastic material of the bag comprises an inner layer and an outer layer. The inner layer comprises polyethylene having a melting point of about 90° C. The outer layer comprises polyamide 6 having a melting point higher than 170° C.

Material can be introduced into the plastic bag through the open top end 12. Once the bag is filled, the bag is closed in such a way that the inner layer is not appearing at the outer surface of the closed bag.

A suitable way to realise this is illustrated in FIGS. 1a to 1e.

The front and back walls of the plastic bag are folded inwards as indicated by arrows 13 and 14. In this way two tips 15 and 16 are formed. In a subsequent step these tips are folded inwards as indicated by arrows 17 and 18.

The folded tips are secured to each other at their contact points 19 for example by means of an adhesive.

The plastic bag is introduced in a disinfecting device and subjected to a heat and compaction treatment.

As the polyethylene has a melting point of about 90° C., which is lower than the disinfecting temperature, the plastic will melt during the disinfecting treatment and will adhere to the waste collected in the plastic bag once the treatment is finished. The polyamide at the other hand, has a higher melting point (higher than 170° C.). Adhesion between the box and the inside of the disinfecting device due to the melting of the polypropylene during the heat treatment is thus avoided. Due to the adhesion between the material collected inside the plastic bag and the polyethylene, the compacted bag will keep its compacted shape after the compaction pressure is released.

FIG. 2 illustrates a method of closing a bag 20 having a straight or slanted flap 21. The plastic bag 20 comprises a front wall 22 and a back wall 24 from which the flap 21 extends.

The bag is closed by folding the flap 22 forwards over the opening 25 of the plastic bag as indicated by arrow 26. The flap can be sealed to the front wall 22 of the plastic bag for example by means of a glue.

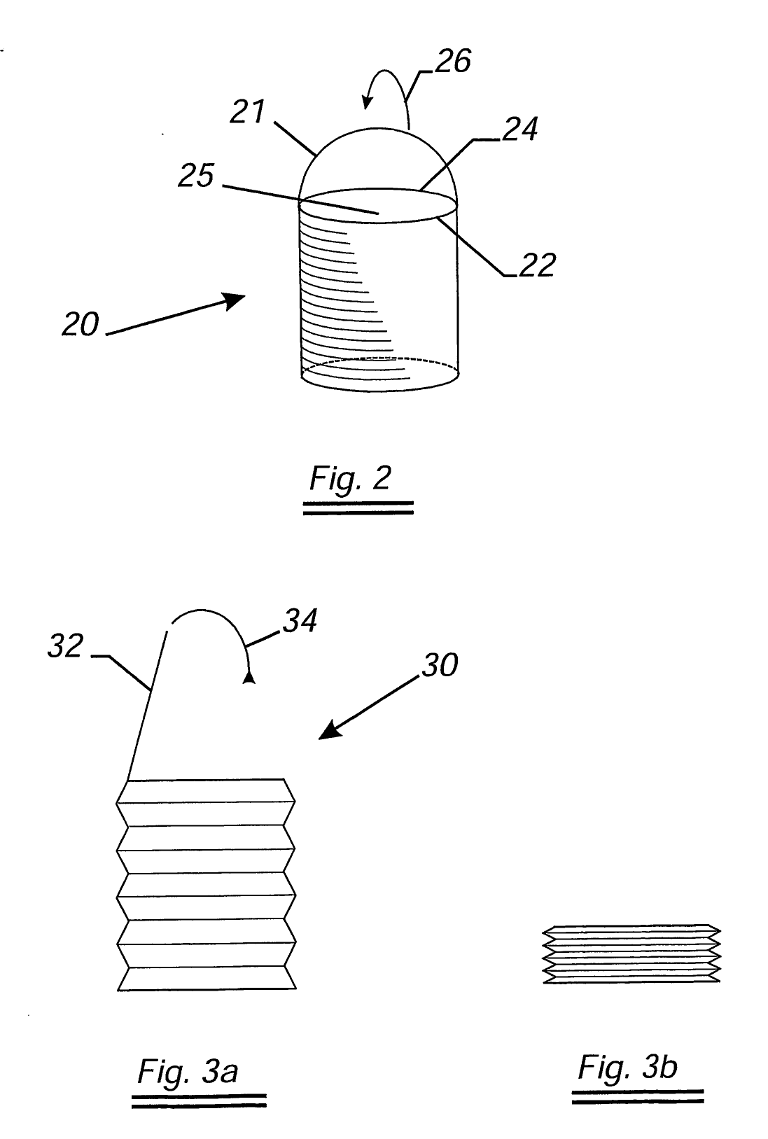

FIG. 3 shows a plastic box 30, provided with a lid 32.

The plastic of the plastic box 30 and of the lid 32 comprises an inner layer and an outer layer. The inner layer comprises polyethylene, the outer layer comprises polypropylene.

Once the box is filled with the waste, for example with needles or other sharp objects, the box is closed by means of the lid.

The box is designed to have a shape that can be easily compacted in a controlled way without creating fractures. The embodiment illustrated in FIG. 3 for example is designed so that it can be folded just like an accordion. FIG. 3b shows the box after the compaction step has been performed.

As the polyethylene has a melting point of about 90° C., which is lower than the disinfecting temperature, the plastic will adhere to the waste collected in the box once the treatment is finished and the compressed box will keep its form after the compaction.

The polypropylene at the other hand, has a higher melting point (between 120° C. and 170° C.). A strong adhesion between the box and the inside of the disinfecting device due to the melting of the polypropylene during the heat treatment is thus avoided.

1. A method of preparing material to be further subjected to a heat treatment, said method comprising the steps of:
introducing said material in a plastic recipient, said plastic recipient comprising an inner layer and an outer layer, said inner layer having a melting point lower than the temperature reached during the heat treatment and said outer layer having a melting point higher than the temperature reached during the heat treatment;
closing said plastic recipient in such a way that the inner layer is not appearing at the outer surface of the closed recipient.

2. A method according to claim 1, whereby said material is subjected to a heat and compaction treatment.

3. A method according to claim 1, whereby said plastic recipient comprises a plastic bag or a plastic box.

4. A method according to claim 1, whereby said inner layer has a melting point lower than the temperature reached during the heat treatment.

5. A method according to claim 1, whereby said outer layer has a melting point higher than the temperature reached during the heat treatment.

6. A method according to claim 1, whereby said inner layer is selected from the group consisting of polyethylene, polycarbonate (PC), polyphenyleneoxide or modified polyphenyleneoxide (PPO), polymethylmethacrylate (PMMA) and polyoxymethylene copolymer.

7. A method according to claim 1, whereby said inner layer is selected from the group consisting of polyamide, polyethyleneterephtalate (PET), silicon, polyvinylidene fluoride (PVDF), polytetrafluoroethylene (PTFE), polyaramide, polyimide (PI), polyetherimide (PI), fluorinated ethylene propylene copolymer (FEP), ethylene tetrafluoroethylene copolymer (ETFE), hexafluoropropylenevinylidenefluoride copolymer (FKM) and tetrafluoroethylene-perfluoro(alkoxy vinyl ether) copolymer.

8. A method according to claim 1, whereby said inner layer has a thickness between 50 and 500 μm.

9. A method according to claim 1, whereby said outer layer has a thickness between 10 and 100 μm.

10. A method according to claim 3, whereby said plastic recipient is provided with a valve.

11. A method according to claim 1, whereby a thermal indicator is added to one or more layers of said plastic recipient.

12. A method according to claim 1, whereby said material comprises medical waste.

13. A method of disinfecting or sterilising material which is prepared according to a method according to claim 1, said method comprising the steps of:
introducing the closed plastic recipient in a disinfecting device;
subjecting the closed plastic recipient to a heat treatment and possibly also to a compaction treatment.

14. A method according to claim 13, said method comprising the steps of:
introducing the closed plastic recipient holding the material to be disinfected in a disinfecting device;
eliminating substantially all the air and free space that is present in and around said material to be disinfected;
establishing airtight conditions;
creating a water vapour pressure and/or radical pressure by heating the receptacle and/or the material.
